(19) United States
(12) Patent Application Publication
(10) Pub. No.: US 2010/0226836 A1
(43) Pub. Date: Sep. 9, 2010

(54) SANITIZER

(76) Inventors: Charles Thur, Broadview Heights, OH (US); David Brickner, Concord, OH (US)

Correspondence Address: FAY SHARPE LLP, 1228 Euclid Avenue, 5th Floor, The Halle Building, Cleveland, OH, 44115, US

(21) Appl. No.: 12/397,522

(22) Filed: Mar. 4, 2009

(51) Int. Cl. A61L 2/04

(52) U.S. Cl. 422/307; 219/400

(57) ABSTRACT

A sanitizing device includes a base, a container supported by the base, a heating element supported by the base, and a blower assembly supported by the base. The container can include a base wall, a side wall, and a hot air inlet vertically offset above the base wall. The blower assembly can be situated with respect to the heating element and the hot air inlet to direct air over the heating element and through the hot air inlet into the container.

### BACKGROUND

Germs can be passed on toys and other moist items, especially moist soft items, since a moist environment is a favorable breeding ground for germs. Sanitizers that employ dry heat to sanitize toys, pacifiers, bottles and other items are known. These known sanitizers typically include a container in which the items to be sanitized are placed. These known sanitizers also include a heating element and a blower assembly. The blower assembly directs air over the heating element and heated air is introduced into the container where the items to be sanitized are placed. Dry heat, as opposed to chemicals, is used to kill bacteria on the items to sanitize these items.

As mentioned above, moist items are often placed in dry heat sanitizers. As these items undergo the sanitizing process, liquid may fall off of the items placed in the sanitizer. Known sanitizers use complicated seals and other mechanisms to handle and direct any liquid that falls off the items that are being sanitized. This can result in complicated construction and assembly.

### SUMMARY

A sanitizing device that can overcome the aforementioned shortcomings, and/or other shortcomings not mentioned above, includes a base, a container supported by the base, a heating element supported by the base, and a blower assembly supported by the base. The container can include a base wall, a side wall, and a hot air inlet formed in the side wall and vertically offset above the base wall. The blower assembly can be situated with respect to the heating element and the hot air inlet to direct air over the heating element and through the hot air inlet into the container. Items that are placed into the container can be sanitized using dry heat.

Another example of a sanitizing device that can overcome the aforementioned shortcomings, and/or other shortcomings not mentioned above, includes a heating element, a container, a blower assembly, and a water-impervious barrier. The container can include a base wall, a side wall and at least one opening formed in the container. The blower assembly can be situated with respect to the heating element and the container to direct air over the heating element and into the container. The water-impervious barrier can extend upwardly from the base wall of the container. The barrier can be configured such that liquid that has fallen from associated articles disposed in the container onto an area of the base wall that is separated from the at least one opening by the barrier must collect enough to flow over the barrier in order to pass through the at least one opening in the container.

### BRIEF DESCRIPTION OF THE DRAWINGS

FIG. 1 is a perspective view of a sanitizing device.

FIG. 2 is an exploded view of the sanitizing device shown in FIG. 1.

FIG. 3 is a top plan view of the sanitizing device shown in FIG. 1 with a lid removed.

### DETAILED DESCRIPTION

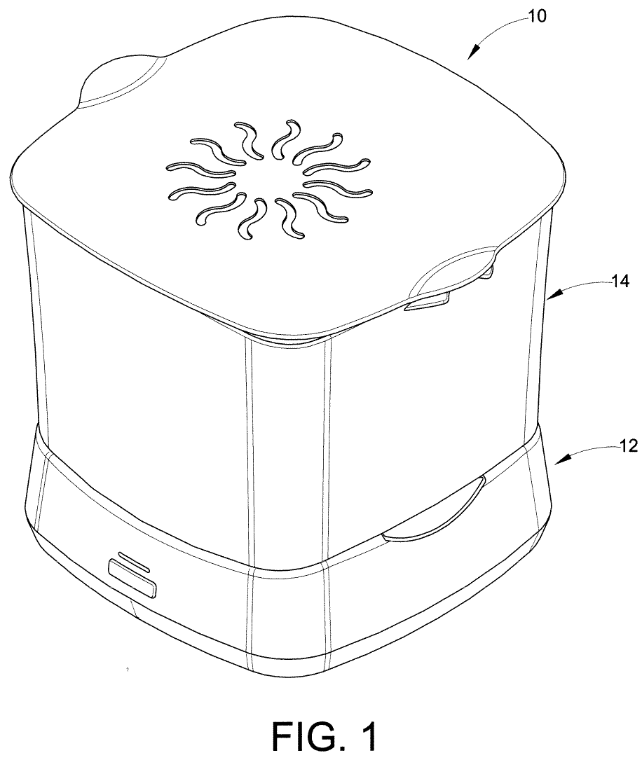

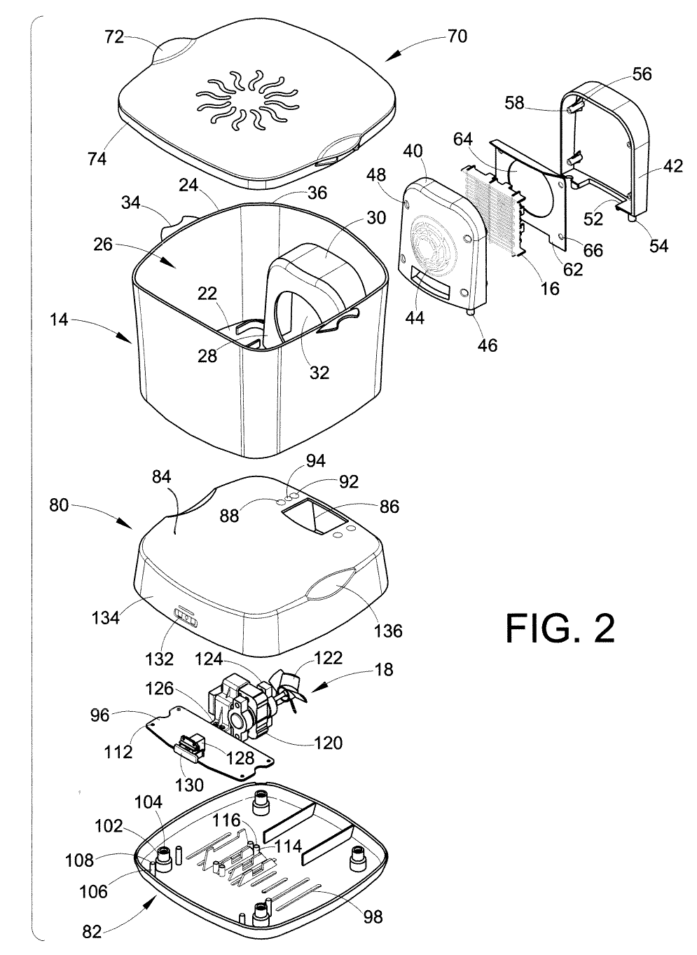

With reference to FIGS. 1 and 2, a sanitizing device 10 includes a base 12, a container 14, a heating element 16, and a blower assembly 18. The sanitizing device is useful to sanitize items using dry heat. The sanitizing device can be used to sanitize hard and soft toys as well as other household items. The items are placed in the container 14, the device 10 is turned ON and heated air is introduced into the container to kill bacteria on the items that are placed in the container.

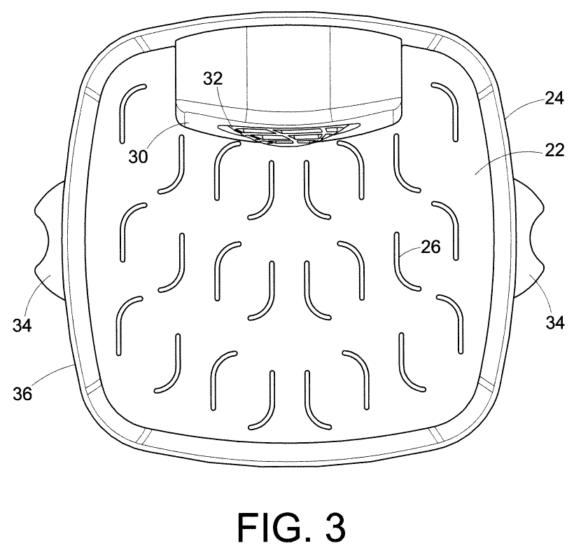

The container 14 can be supported by the base 12. With reference to FIGS. 2 and 3, the container 14 can include a base wall 22 and a side wall 24 that extends upwardly from the base wall. In the depicted embodiment, the side wall 24 of the container 14 extends substantially normal to the base wall 22 and extends upwardly from a periphery of the base wall to define an enclosed space 26 in which items to be sanitized can be placed. In the depicted embodiment, the container 14 is made from a material that is impervious to water, for example a durable plastic material. As more clearly seen in FIG. 3, for the depicted embodiment the base wall 22 is devoid of any openings and includes a plurality of ribs 26 integrally formed with and extending upwardly from the base wall. In the embodiment depicted in FIGS. 1-3, each rib 26 is offset from the side wall 24. The ribs 26 space the articles placed into the container 14 from the base wall 22 so that air can flow underneath and around the items placed inside the container 14 to encourage warm air to contact the greatest surface area as possible for the articles that are disposed in the container. Since the container 14 is made from a material that is impervious to water and the base wall 22 of the container is devoid of openings within the area of the base wall that is surrounded by the upwardly extending side wall 24, liquid falling from articles disposed in the container is precluded from falling on the heating element 16, the blower assembly 18 and other electrical components found in the sanitizing device.

The container 14 in the depicted embodiment also includes an indented section 30. In the depicted embodiment, the indented section 30 extends inwardly from a portion of the side wall 24. More particular to the depicted embodiment, the container 14 has four sides and the indented section extends inwardly into the enclosed space 26 from one of the four sides. Alternatively, an indented section could extend upwardly from the base 22 and be offset from a side of the container 14, if desired. With reference back to the depicted embodiment, the indented section 30 can be referred to as a portion of the side wall 24 of the container 14 and defines a hot air inlet 32, which is an opening through the side wall at the indented section. The hot air inlet 32 is vertically offset above the base wall 22. Because of this configuration, the portion of the side wall 24 of the container 14 disposed below the hot air inlet 32 can be referred to as a water-impervious barrier 28 that extends upwardly from the base wall 22 of the container adjacent the hot air inlet. This barrier 28 is configured so that liquid falling from moist articles disposed in the container 14 must puddle on the base wall 22 and collect enough to flow over the barrier in order to pass through the air inlet 32. The barrier 28 can preclude liquid that has fallen from the moist articles found in the container 14 from traveling towards the heating element 16, the blower assembly 18, and the electrical components that are connected with the heating element and the blower assembly. In the depicted embodiment, the heating element 16 is disposed vertically above or is at the same elevation as the base wall 22 of the container 14. This makes it difficult for any liquid that may collect in the container 14 from falling on the heating element.

In an alternative embodiment, for example where the indented section can extend upwardly from the base wall of the container and be offset from a side of the container, a water-impervious barrier (similar to the barrier 28) can extend upwardly from the base wall adjacent at least one opening in the container. This at least one opening would be similar to the hot air inlet 32 described above. This barrier is configured such that liquid falling from associated articles disposed in the container must puddle on the base wall and collect enough liquid to flow over the barrier in order to pass through the at least one opening in the container. In an alternative embodiment where an opening is defined in the base of the container and the heating element is received within this opening, e.g. there is no indented section in the container, a water-impervious barrier (similar to the barrier 28) can also extend upwardly from the base wall adjacent at least one opening in the container. This barrier can also be configured such that liquid that has fallen from associated articles disposed in the container must puddle on the base wall and connect enough liquid to flow over the barrier in order to pass through the at least one opening in the container. In each of the aforementioned alternative embodiments, the base wall of the container can be devoid of any openings with the exception of the at least one opening. The barrier can be configured such that liquid that has fallen from articles disposed in the container onto an area of the base wall that is separated from the at least one opening by the barrier must collect enough to flow over the barrier in order to pass through the at least one opening in the container.

With continued reference to FIG. 2, the indented section 30 can provide a location for the heating element 16 and can also operate as an indexing feature for the container for when the container is removed from and reattached with the base 12. In the embodiment depicted in FIGS. 1-3, an uppermost portion of the indented section 30 is vertically below an upper edge 36 of the side wall 24. Handle ledges 34 (two are shown) are formed on opposite exterior sides of the side wall 24 adjacent the upper edge 36. The handle ledges 34 can be gripped by an operator of the device 10 to remove the container 12 from the base 14 without damaging the base or the container.

With reference to FIG. 2, in the depicted embodiment the heating element 16 is disposed within a heating element shell. In the embodiment depicted in FIG. 2, the heating element shell can be supported by the base 12 and be disposed in the indented section 30 outside of the container 14. The heating element 16 can be disposed in the heating element shell and the heating element shell can have a configuration that generally complements the indented section 30, e.g. the external configuration of the heating element shell ban generally match the internal configuration of the indented section. Moreover, an external surface of the heating element shell can be generally flush with an external surface of the side of the container 14. As shown in FIG. 2, the heating element shell includes a front housing 40 and a rear housing 42. The front housing 40 generally defines a hot air exit 44, which in the depicted embodiment is a plurality of openings, for the heating element shell. The hot air exit 44 can align with the hot air inlet 32 of the container 14. The front housing 40 also includes vertically oriented downwardly depending posts 46 and horizontally extending fastener openings 48.

The heating element shell also includes the rear housing 42, which along with the front housing 40 can define a cool air inlet 52. The cool air inlet 52 in the depicted embodiment is formed in a lower section of the front housing 40 and the rear housing 42 and is disposed beneath the heating element 16. The rear housing 42 also includes vertically oriented downwardly depending mounting posts 54, which are similar to the mounting posts 46 formed on the front housing 40. The rear housing 42 also includes horizontally oriented standoffs 56, which each include a respective fastener opening 58 that align with the fastener openings 48 in the front housing 40 to connect the front housing with the rear housing using a fastener (not shown).

The heating element 16 is sandwiched between the front housing 40 and the rear housing 42. An air guide 62 is also sandwiched between the front housing 40 and the rear housing 42. In the depicted embodiment, the air guide 62 is disposed upstream from the heating element 16. The air guide 62 is a plate-like member that includes a large circular central opening 64 through which air can flow en route to the heating element 16 and a plurality of fastener openings 66 that align with the fastener openings 48 and 58 to connect the air guide with the front housing 40 and rear housing 42, respectively. In the depicted embodiment, the heating element 16 is a resistive-type heating element.

With continued reference to FIG. 2, the sanitizing device 10 also includes a lid 70 that can cover the container 14 to further define the enclosed space 26 in which articles to be sanitized are placed. In the depicted embodiment, the lid 70 includes wings 72 (two are shown) that align with the handle ledges 34 when the lid 70 is placed on the container 14. The wings 72 provide a location for an operator of the sanitizing device to place his fingers under the lid to remove the lid from the container. The lid 70 also includes a downwardly depending apron 74 that is internally offset from a peripheral edge of the lid to provide a locating feature for placing the lid on the container. The lid 70 also includes a plurality of vents 76, which allows hot air to leave the enclosed space 26.

The container 14 and the heating element shell 40, 42 are supported by the base 12. As mentioned above, the container 14 is removable from the base 12 without damaging the base or the container. When the container 14 is removed from the base 12 the heating element 16 and the heating element shell 40, 42 remain supported by and connected to the base. The base includes an upper housing 80 and a lower housing 82 connected with the upper housing. The base encloses the blower assembly 18 and other electrical components, which will be described in more detail below.

The upper housing 80 of the base 12 includes an upper wall 84. In the depicted embodiment, the heating element 16 is disposed vertically above the upper wall 84 of the base 12. The upper wall 84 defines a cool air outlet 86. The cool air outlet 86 formed in the upper wall 84 communicates with the cool air inlet 52 of the heating element shell 40, 42 to allow air to pass into the heating element shell. The cool air outlet 86 is disposed on an external side of the side wall 24 of the container 14, and more particularly on an external side of the indented section 30, with respect to the enclosed space 26. The water-impervious barrier 28, which extends upwardly from the base wall 22 of the container 14, prevents liquid from falling through the cool air outlet 86, unless enough liquid collects on the base wall 22 to flow over this water-impervious barrier. This can protect the electrical components that are housed in the base 12. As is more clearly seen in FIG. 2, a majority of the surface area of the upper wall 84 of the upper housing 80 of the base 12 is devoid of any openings and is impervious to water. This can provide protection for the electrical components found in the base 12.

The upper wall 84 is formed to include inwardly disposed post receiving openings 88 and outwardly disposed post receiving openings 92. The inwardly disposed post receiving openings 88 receive the posts 46 that depend downwardly from the front housing 40 of the heating element shell. The outwardly disposed post receiving openings 92 receive the downwardly depending posts 54 that depend from the rear housing 42. Accordingly, the heating element shell connects with the base 12 by the posts attaching in the respective openings. Fasteners can also be used to provide a more secure attachment.

A wire conductor opening 94 can also be provided in the upper wall 84 of the upper housing 80 between a set of the inwardly disposed post receiving openings 88 and the outwardly disposed post receiving openings 92. The wire conductor opening 94 can receive a wire (not shown) to provide for an electrical connection to the heating element 16 in the heating element shell. The wire (not shown) can attach to a printed circuit board (“PCB”) 96, which can be disposed inside the base 12. The PCB 96 can be in electrical communication with a power source (not shown) via an electrical plug and cord (also not shown). The PCB 96 can provide an electrical path from an associated power source, which could be any AC or DC power source, to the blower assembly 18 and the heating element 16. The cool air outlet 86 in the upper wall 84 of the base 12 can be horizontally offset from the PCB 96 and the area of the upper wall 84 directly vertically above the circuit board can be devoid of openings and impervious to water.

The lower housing 82 of the base 12 includes a plurality of inlet vents 98 through which air is drawn by the blower assembly 18. The lower housing 82 also includes upper housing standoffs 102 each including a respective opening 104 to receive a fastener (not shown) to connect the upper housing 80 to the lower housing 82. The lower housing 82 also includes a plurality of PCB standoffs 106 which each include a respective opening 108. The PCB 96 includes a plurality of openings 112 that align with the openings 108 on the PCB standoffs 106 to fasten the PCB to the lower housing 82. The lower housing 82 also includes a plurality of motor mount standoffs 114 each including a respective opening 116.

The blower assembly 18 includes a motor 120, an impeller 122 connected with the motor, and a motor mount 124 inside the base. The blower assembly can be disposed below the upper wall 84 of the base 12. The motor mount 124 in the depicted embodiment is generally U-shaped and includes openings 126 that align with the openings 116 in the motor mount standoffs 114 to connect the motor mount to the lower base housing 82 and thus mount the blower assembly 18 within the base 12.

An ON/OFF switch 128 mounts to the PCB 96. The ON/OFF switch controls the delivery of power to the motor 120 of the blower assembly 18 and can also control the delivery of power to the heating element 16. Circuitry can be provided on the PCB 96 to limit the amount of time that power is delivered to either the motor 120 or the heating element 16. For example, a timing circuit (not shown) can be provided so that the sanitizing device 10 only operates for a predetermined number of minutes before power is no longer delivered to the motor 120 or the heating element 16. The ON/OFF switch can be operated by a button 130 that can extend through an opening 132 formed in an apron 134 that depends downwardly from the upper wall 84 of the upper housing 80 of the base 12. Recesses 136 (two are shown) can be formed where the apron 134 meets the upper wall 84 in the upper housing 80 of the base 12 to provide a location where an operator of the sanitizing device 10 can grip the container 14 for removal from the base 12.

To operate the device, an operator places the container 14 onto the base 12. Since the heating element shell 40, 42 is connected with the base 12 and the indented section 30 is provided in the container 14, locating the container 14 on the base 12 is facilitated by the indexing feature that the heating element shell provides. With the lid 70 removed from the container 14, items to be sanitized can be loaded into the enclosed space 26 defined by the container 14 and the lid 70. The lid 70 can then be placed on top of the container 14. The operator can then depress the button 130 which activates the ON/OFF switch 128 to deliver power to the motor 120 and the heating element 16. As the impeller 122 of the blower assembly rotates 18 because of its connection to the motor 120, air is drawn through air inlets 98 formed in the lower housing 82 of the base 12. The impeller 122 pushes this cool air through the cool air outlet 86 formed in the upper wall 84 of the upper housing 80 of the base 12. Cool air then enters the heating element shell 40, 42 through the cool air inlet 52. Cool air passes through the central opening 64 of the air guide 62 and then over the heating element 16. Hot air exits the heating element shell 40, 42 through the hot air exit 44 and then enters into the enclosed space 26 of the container 14 through the hot air inlet 32. Hot air can then exit the enclosed space 26 through vent 76 formed in the lid 70 en route to ambient.

A sanitizing device and alternatives thereof have been described in detail above. Modifications and alterations will come to those skilled in the art upon reading and understanding the preceding detailed description. The invention is not limited to only those embodiments that are disclosed above. Instead, the invention is broadly defined by the appended claims and the equivalents thereof.

1. A sanitizing device comprising:
a base;
a container supported by the base and including a base wall, a side wall and a hot air inlet formed in the side wall and vertically offset above the base wall;
a heating element supported by the base; and
a blower assembly supported by the base, the blower assembly being situated with respect to the heating element and the hot air inlet to direct air over the heating element and through the hot air inlet into the container.

2. The device of claim 1, further comprising a lid covering the container to define an enclosed space, the lid including vents.

3. The device of claim 1, wherein side wall of the container includes an indented section that extends inwardly into the container, and the heating element is disposed in the indented section.

4. The device of claim 1, wherein the container includes an indented section that extends inwardly into the container, and the heating element is disposed in the indented section.

5. The device of claim 4, further comprising a heating element shell supported by the base and disposed in the indented section outside of the container, the heating element being disposed in the heating element shell and the heating element shell have a configuration that generally complements the indented section.

6. The device of claim 5, wherein the container is removable from the base without damaging the base or the container, when the container is removed from the base the heating element and the heating element shell remains supported by the base.

7. The device of claim 6, wherein the heating element shell includes a cool air inlet and a hot air exit, and wherein the base includes a cool air outlet aligned with the cool air inlet.

8. The device of claim 7, wherein the hot air exit aligns with the hot air inlet of the container.

9. The device of claim 8, wherein the base includes an upper wall that defines the cool air outlet being disposed on an external side of the side wall of the container.

10. The device of claim 9, wherein an upper edge of the heating element shell is disposed vertically below an upper edge of the container.

11. The device of claim 9, further comprising a circuit board for providing an electrical path from an associated power source to the blower assembly and the heating element, the cool air outlet in the upper wall of the base being horizontally offset from the circuit board and a portion of the upper wall directly vertically above the circuit board being impervious to water.

12. The device of claim 11, wherein a majority of the surface area of the upper wall of the base is devoid of openings and is impervious to water.

13. The device of claim 12, wherein the blower assembly includes a motor and an impeller, the motor and the impeller being disposed below the upper wall of the base.

14. The device of claim 13, wherein the heating element is disposed vertically above the upper wall of the base.

15. The device of claim 1, wherein the container includes a plurality of ribs integrally formed with and extending upwardly from the base wall.

16. The device of claim 15, wherein each rib is offset from the side wall.

17. A sanitizing device comprising:
a heating element;
a container including a base wall, a side wall and at least one opening formed in the container;
a blower assembly situated with respect to the heating element and the container to direct air over the heating element and into the container; and
a water-impervious barrier extending upwardly from the base wall of the container adjacent the at least one opening, barrier being configured such that liquid that has fallen from associated articles disposed in the container onto an area of the base wall that is separated from the at least one opening by the barrier must collect enough to flow over the barrier in order to pass through the at least one opening in the container.

18. The device of claim 17, wherein the water impervious barrier is a portion of the side wall.

19. The device of claim 17, wherein the base wall of the container is devoid of any openings with the exception of the at least one opening.

20. The device of claim 17, wherein the base wall of the container is devoid of any openings within the area of the base wall that is bounded by the side wall.
